(19) United States
(12) Patent Application Publication
(10) Pub. No.: US 2003/0118542 A1
(43) Pub. Date: Jun. 26, 2003

(54) Film-forming cosmetic composition

(76) Inventors: Frederic Auguste, Chevilly-Larue (FR); Valerie De La Poterie, le Chatelet en Brie (FR); Emmanuelle Portois, Chatillon (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/271,974

(22) Filed: Oct. 17, 2002

(30) Foreign Application Priority Data
Oct. 17, 2001 (FR) 01 13380

(51) Int. Cl. A61K 7/021; A61K 7/075; A61K 7/08

(52) U.S. Cl. 424/70.22

(57) ABSTRACT

A composition comprising, in a physiologically acceptable medium, at least one hydrophobic film-forming polymer and at least one ionic surfactant exhibiting a variation in conductivity in water, at a concentration of 2% by weight in water, of at least 100 μS/cm in a temperature range of from 20° C. to 45° C., for producing a composition, such as a film, deposited on a keratin material, which can be removed as make-up from the keratin material with hot water. The application of the make-up composition to, and the care of a keratin material.

The present invention relates, for example, to the inclusion of at least one hydrophobic film-forming polymer and at least one particular surfactant in, or for the manufacture of, a composition for producing a deposit, such as a film, that can be removed, for example as make-up from a keratin material, with hot water.

The present invention also relates, for example, to a care or make-up composition for a keratin material, for example, the skin, the eyelashes, the eyebrows, the hair and the nails, such as of human beings, comprising at least one hydrophobic film-forming polymer and at least one particular surfactant, and a method for the cosmetic care of or the application of make-up to the keratin material.

The composition may be provided in a variety of forms, for example, mascara, eyeliner, product for lips, blusher or eyeshadow, foundation, make-up product for a body, concealer, product for a nail, composition for protecting against sunlight or for colouring skin, and a care product for the skin. In one embodiment of the present invention, the composition is provided in the form of a mascara.

The document WO-A-95/15741 discloses mascara compositions in the form of a wax-in-water emulsion comprising surfactants. However, the make-up film obtained with such compositions may not have good resistance to water, thus the film partially disintegrates by crumbling or spreading around the eye on contact with water, for example, during bathing or showering. The disintegration of the film can cause substantial loss of the intensity of the colour of the make-up, which forces the consumer to repeat the application of the mascara. The spreading of the film can form a halo around the area where make-up has been applied, which is not aesthetic. Tears and perspiration can also cause these same disadvantages.

To enhance the water resistance of the make-up, it is known from the document U.S. Pat. No. 4,423,031 to use acrylic polymers in aqueous dispersion. The mascara of such composition can be difficult to remove as make-up and requires the use of specific make-up removers based on oils or organic solvents. However, these make-up removers may irritate the eye, may cause, for example, pricklings, may leave a film over the eye, or may leave an uncomfortable fatty residual film on the skin around the eye and eyelids.

To avoid the use of those specific make-up removers, water and soap can be used as described in the document WO-A-96/33690 for a mascara comprising a water-insoluble polymer and a water-soluble film-forming polymer. However, the use of soap may cause ocular discomfort due to pricklings or to the deposition of a film over the eye. Furthermore, the soap can solubilize the make-up film, which then spreads around the eye forming unattractive halos and staining the skin.

The use of hot water, i.e., water having a temperature greater than or equal to 35° C. (temperature measured at atmospheric pressure), and, for example, ranging approximately from 35° C. to 50° C., makes it possible to avoid the disadvantages of the make-up removers known in the art. However, the mascara compositions resistant to cold water as described above cannot be removed with hot water.

The present invention can, for example, provide a cosmetic composition for deposit on a keratin material, wherein the deposited composition can be removed as make-up using hot water while maintaining good resistance to cold water.

The inventors have created such a composition comprising at least one film-forming polymer capable of forming a hydrophobic film and at least one particular surfactant. After applying the composition to a keratin material, for example, the eyelashes, the make-up obtained can be quite resistant to cold water, i.e., water having a temperature of less than or equal to 30° C., for example, water during bathing, to tears and/or to perspiration. Such make-up can be easily removed with hot water, for example, by rubbing with cotton wool or a gauze: such make-up can peel off easily from the eyelashes and can be removed from the eyelashes without fragmenting (in the form of a coating layer) or in the form of fragments or pieces. Thus, such removed make-up does not spread on the skin, the skin remains clean, avoiding the formation of halos around the eye and staining of the skin. Such make-up can be very simply removed with hot water, such as hot water containing no detergent agent such as soaps. For the make-up removal, the hot water used may be chosen from tap water, demineralized water, and mineral water at a temperature of greater than or equal to 35° C., for example a temperature ranging from approximately 35° C. to 50° C..

An embodiment of the invention is a composition comprising, in a physiologically acceptable medium, at least one hydrophobic film-forming polymer and at least one ionic surfactant, exhibiting a variation in conductivity in water at a concentration of 2% by weight in water, of at least 100 μS/cm in a temperature range of from 20° C. to 45° C., the composition being able to provide a film deposited on a keratin material, such film being removable as make-up with hot water. Another embodiment of the invention is a make-up composition for deposit on a keratin material, comprising in said composition, a physiologically acceptable medium, at least one hydrophobic film-forming polymer, and at least one ionic surfactant exhibiting a variation in conductivity in water, at a concentration of 2% by weight in water, of at least 100 μS/cm in a temperature range of from 20° C. to 45° C., wherein said deposited composition is removable from the keratin material with hot water. Another embodiment of the invention is a method for application of a make-up to, or non-therapeutic care of, a keratin material comprising depositing on the keratin material a composition comprising, in a physiologically acceptable medium, at least one hydrophobic film-forming polymer and at least one ionic surfactant exhibiting a variation in conductivity in water, at a concentration of 2% by weight in water, of at least 100 μS/cm in a temperature range of from 20° C. to 45° C., wherein the deposited composition is removable with hot water

The term “physiologically acceptable medium” is understood to mean a medium compatible with keratin materials, for example, a cosmetic medium.

The composition can contain little, or be free of, additional surfactant different from the said ionic surfactant defined above, for example, in an amount of less than 0.5% by weight relative to the total weight of the composition. In such a case, the composition exhibits good resistance to cold water.

The term “additional surfactant” means any amphiphilic compound chosen from nonionic amphiphilic compounds having an HLB (hydrophilic-lipophilic balance) greater than or equal to 10, and the ionic amphiphilic compounds in which the hydrophilic part comprises a counter-ion having a molar mass greater than or equal to 50 g/mol, these ionic amphiphilic compounds being different from the said ionic surfactant defined above.

The removal of deposited composition from a keratin material, such as removal of film as a make-up, with hot water can be obtained by using an ionic surfactant, at a concentration of 2% by weight in water, exhibiting a variation in conductivity in water of at least 100 μS/cm in a temperature ranging from 20° C. to 45° C., for example, a temperature ranging from 30° C. to 45° C., and for further example, a temperature ranging from 40° C. to 45° C. In one embodiment of the present invention, the said variation in conductivity ranges from 100 to 2000 μS/cm, for example said variation in conductivity may range from 200 to 1500 μS/cm, and as a further example, said variation in conductivity may range from 200 to 1000 μS/cm.

Such ionic surfactant can make the deposited composition, such as polymer film, more sensitive to water. Thus, for example, such make-up film is embrittled during contact with hot water, and the film disintegrates easily and becomes detached from its support by rubbing, for example, with the fingers, a glove, or cotton wool.

In one embodiment of the invention, the said ionic surfactant is an anionic surfactant. The anionic surfactant may be chosen from sodium stearate, potassium stearate, and the phthalamates of formula (I):

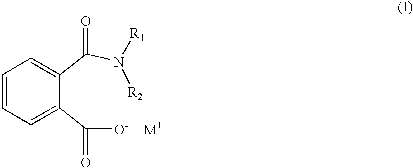

in which R₁ is chosen from alkyl radicals having from 16 to 20 carbon atoms; R₂ is chosen from H and —CH₃; and M is chosen from H, Na, K, NH₄, (HOCH₂CH₂)₃NH, and (HOCH₂CH₂)₂NH₂. In another embodiment, R₁ is chosen from alkyl radicals having 18 carbon atoms. In yet another embodiment, R₂ is a hydrogen atom.

The at least one ionic surfactant is, for example, chosen from sodium stearate and sodium N-octadecylphthalamate. In one embodiment, the at least one ionic surfactant is sodium N-octadecylphthalamate.

The at least one ionic surfactant has a molecular weight ranging, for example, from 200 to 1000, further, for example from 250 to 800, and for further example from 250 to 600. (The molecular weight of sodium stearate is 306 and the molecular weight of sodium N-octadecylphthalamate is 440).

Accordingly, another aspect of the invention is a composition comprising, in a physiologically acceptable medium, at least one hydrophobic film-forming polymer and at least one ionic surfactant of formula (I):

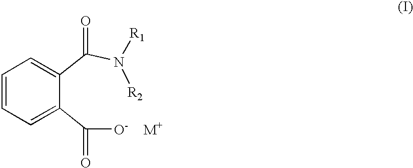

in which R₁ is chosen from alkyl radicals having from 16 to 20 carbon atoms; R₂ is chosen from H and —CH₃; M is chosen from H, Na, K, NH₄, (HOCH₂CH₂)₃NH, and (HOCH₂CH₂)₂NH₂. In one embodiment, R₁ is chosen from alkyl radicals having 18 carbon atoms. In another embodiment, R₂ is a hydrogen atom.

The at least one ionic surfactant may be present in the composition in an amount ranging, for example, from 0.1% to 20% by weight relative to the total weight of the composition, and further for example, from 0.5% to 15% by weight relative to the total weight of the composition, and still further, for example, from 1% to 10% by weight relative to the total weight of the composition.

In another embodiment, the composition may further comprise glyceryl monostearate in an amount ranging, for example, from 0.1% to 5% by weight relative to the total weight of the composition, and further for example from 0.5% to 2% by weight relative to the total weight of the composition. The at least one ionic surfactant, combined with glyceryl monostearate, can produce a variation in the conductivity described above for this mixture that is much greater than that of the said ionic surfactant alone and thus results in an even easier removal as make-up with hot water of the film deposited on the keratin materials.

In one embodiment, the at least one ionic surfactant and the glyceryl monostearate may be present in the composition in a ratio of ionic surfactant/glyceryl monostearate by weight ranging, for example, from 0.01:1 to 2:1, and further for example, from 0.5:1 to 2:1, and still further for example from 1:1 to 2:1.

The composition according to the invention may also comprise at least one film-forming polymer capable of forming a hydrophobic film. In the present application, the term “film-forming polymer” means a polymer capable of forming, on its own or in the presence of a film-forming aid, a continuous and adherent film on a support, such as on a keratin material.

The term “film-forming polymer capable of forming a hydrophobic film” means a polymer whose film has a solubility in water at 25° C. of less than 1% by weight relative to the total weight of the film.

The film-forming polymer may be chosen from synthetic polymers, for example, free-radical polymers, polycondensates, polymers of natural origin, and mixtures thereof.

The term “free-radical film-forming polymer” means a polymer obtained by polymerization of monomers with, for example, ethylenic unsaturation (in contrast to polycondensates).

The free-radical film-forming polymers may be chosen from, for example, vinyl polymers and copolymers, such as acrylic polymers.

The vinyl film-forming polymers may result from the polymerization of ethylenically unsaturated monomers having at least one acid group, esters of these acid monomers, and/or amides of these acid monomers.

The monomer carrying at least one acid group may be chosen, for example, from α,β-ethylenic unsaturated carboxylic acids, for example acrylic acid, (meth)acrylic acid, crotonic acid, maleic acid and itaconic acid.

The esters of the acid monomers may be, for example, chosen from the esters of (meth)acrylic acid (also called (meth)acrylates), for example, alkyl (meth)acrylates, such as C₁-C₃₀ alkyl (meth)acrylates, and further such as C₁-C₂₀ alkyl (meth)acrylates, which may be linear, branched, or cyclic, aryl (meth)acrylates, for example, C₆-C₁₀ aryl (meth)acrylates, and hydroxyalkyl (meth)acrylates, such as C₂-C₆ hydroxyalkyl (meth)acrylates.

Among the alkyl (meth)acrylates, mention may be made of methyl methacrylate, ethyl methacrylate, butyl methacrylate, isobutyl methacrylate, 2-ethylhexyl methacrylate, lauryl methacrylate and cyclohexyl (meth)acrylate.

Among the hydroxyalkyl (meth)acrylates, mention may be made of hydroxyethyl acrylate, 2-hydroxypropyl acrylate, hydroxyethyl methacrylate and 2-hydroxypropyl methacrylate.

Among the aryl (meth)acrylates, mention may be made of benzyl acrylate and phenyl acrylate.

In one embodiment of the present invention, the esters of (meth)acrylic acid are chosen from alkyl (meth)acrylates.

According to the present invention, the alkyl group of the esters may be either fluorinated or perfluorinated, i.e., some or all of the hydrogen atoms of the alkyl group may be substituted with fluorine atoms.

Among the amides of the acid monomers, mention may be made, for example, of (meth)acrylamides, such as N-alkyl(meth)acrylamides, for example, the N-alkyl(meth)acrylamides with the alkyl group chosen from C₁-C₂₀ alkyl groups. Among the N-alkyl(meth)acrylamides, mention may be made of N-ethylacrylamide, N-t-butylacrylamide, N-t-octylacrylamide and N-undecylacrylamide.

The vinyl film-forming polymers may also result from the homopolymerization or copolymerization of at least one monomer chosen from vinyl esters, olefins (including fluorinated olefins), vinyl ethers and styrene monomers. For example, such monomers may be polymerized with acid monomers, esters of the acid monomers, and/or amides of the acid monomers, such as those mentioned above.

Among the vinyl esters, mention may be made of vinyl acetate, vinyl neodecanoate, vinyl pivalate, vinylbenzoate and vinyl t-butyl benzoate.

Among the olefins, mention may be made of ethylene, propylene, butene, isobutene, octene, octadecene, and fluorinated polyolefins, for example tetrafluoroethylene, vinylidene fluoride, hexafluoropropene, and chlorotrifluoroethylene.

Among the styrene monomers, mention may be made of styrene and alpha-methylstyrene.

In one embodiment of the invention, the film-forming polymer may be chosen from polycondensates, for example, polyurethanes, such as anionic, cationic, nonionic or amphoteric polyurethanes, polyurethane-acrylics, polyurethane-polyvinylpyrrolidones, polyester-polyurethanes, polyether-polyurethanes, polyureas, polyurea-polyurethanes, and mixtures thereof.

The film-forming polyurethane may be chosen from, for example, aliphatic, cyclic and aromatic polyurethane, polyurea-urethane and polyurea copolymers comprising alone or as a mixture:

at least one block of aliphatic, cyclic and/or aromatic origin,

at least one branched or unbranched silicone-containing block, for example polydimethylsiloxane or polymethylphenylsiloxane, and/or

at least one block comprising fluorinated groups.

Among the film-forming polycondensates, mention may be made of polyesters, polyester amides, polyesters with a fatty chain, polyamides and epoxy ester resins, resins resulting from the condensation of formaldehyde with an arylsulphonamide, and arylsulphonamide epoxy resins.

The polyesters may be obtained, in a manner known in the art, by polycondensation of dicarboxylic acids with polyols, for example, diols.

Such dicarboxylic acids may be aliphatic, alicyclic or aromatic, for example: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, diglycolic acid, thiodipropionic acid, 2,5-naphthalenedicarboxylic acid and 2,6-naphthalenedicarboxylic acid. Such dicarboxylic acid monomers may be used alone or in combination with at least two dicarboxylic acid monomers. Among these monomers, mention may be made of phthalic acid, isophthalic acid, and terephthalic acid.

The diols may be chosen from aliphatic, alicyclic and aromatic diols, for example: ethylene glycol, diethylene glycol, triethylene glycol, 1,3-propanediol, cyclohexanedimethanol and 4-butanediol. Among other polyols, mention may be made of, for example, glycerol, pentaerythritol, sorbitol, and trimethylolpropane.

The polyester amides may be obtained in a manner similar to the polyesters, by polycondensation of diacids with diamines or amino alcohols. Such diamines may be chosen from ethylenediamine, hexamethylene-diamine, and meta- and para-phenylenediamine. Mention may be made of monoethanolamine as such aminoalcohol.

The polyester may further comprise at least one monomer residue carrying at least one —SO₃M group, wherein M is chosen from a hydrogen atom, an ammonium ion (NH₄⁺) and a metal ion, for example, an Na⁺, Li⁺, K⁺, Mg²⁺, Ca²⁺, Cu²⁺, Fe²⁺ or Fe³⁺ ion. Mention may be made of a bifunctional aromatic monomer comprising such an —SO₃M group, wherein the aromatic ring of such bifunctional aromatic monomer may be chosen from, for example benzene, naphthalene, anthracene, diphenyl, oxydiphenyl, sulphonyldiphenyl and methylenediphenyl rings. Mention may further be made of the bifunctional aromatic monomer carrying, in addition, an —SO₃M group, for example, sulphoisophthalic acid, sulphoterephthalic acid, sulphophthalic acid, and 4-sulphonaphthalene-2,7-dicarboxylic acid.

In one embodiment of the present invention, the copolymers based on isophthalate/sulphoisophthalate, for example, copolymers obtained by condensation of diethylene glycol, cyclohexanedimethanol, isophthalic acid and sulphoisophthalic acid can be used. Such polymers are sold, for example, under the trade name Eastman AQ by the company Eastman Chemical Products.

The synthetic hydrophobic polymer may also be a silicone polymer chosen from, for example, silicone polymers of the polyorganopolysiloxane type.

The optionally modified polymers of natural origin may be chosen from shellac resin, sandarac gum, dammars, elemis, copals, and cellulose polymers, for example nitrocellulose, cellulose acetate, cellulose acetobutyrate, cellulose acetopropionate, ethyl cellulose, and mixtures thereof.

In one embodiment of the invention, the film-forming polymer may be present in the form of solid particles dispersed in an aqueous medium. The term polymer in the form of solid particles in aqueous dispersion, generally known by the name of latex or pseudolatex, means a phase containing water and optionally a compound soluble in water, in which the polymer is directly dispersed in the form of particles.

The size of the particles of polymers in aqueous dispersion may range for example, from 10 nm to 500 nm, and also for example from 20 nm to 300 nm.

The aqueous medium may consist essentially of water or alternatively may also comprise a mixture of water and a water-miscible solvent, for example lower monoalcohols having from 1 to 5 carbon atoms, glycols having from 2 to 8 carbon atoms, C₃-C₄ ketones, and C₂-C₄ aldehydes. It represents, for example, from 5% to 94.9% by weight relative to the total weight of the composition.

Among the film-forming polymer in aqueous dispersion, mention may be made of acrylic polymers, for example, sold under the names NEOCRYL XK-90®, NEOCRYL A-1070®, NEOCRYL A-1090®, NEOCRYL BT-62®, NEOCRYL A-1079®, NEOCRYL A-523® by the company AVECIA-NEORESINS, DOW LATEX 432® by the company DOW CHEMICAL; and polyurethanes, for example, the polyester-polyurethanes sold under the names “AVALURE UR-405®”, “AVALURE UR-410®”, “AVALURE UR-425®”, “SANCURE 2060®” by the company GOODRICH, the polyether-polyurethanes sold under the names “SANCURE 878®”, “AVALURE UR-450®” by the company GOODRICH, “NEOREZ R 970®” by the company ICI, and the polyurethanes-acrylics sold under the name NEOREZ R-989® by the company AVECIA-NEORESINS.

Alkali-soluble polymers may also be used, wherein the pH of the composition is adjusted so as to maintain these polymers in the state of particles in aqueous dispersion.

The composition according to the invention may further comprise a film-forming aid promoting the formation of a film with the particles of the film-forming polymer. Such a film-forming aid may be chosen from all the compounds known to a person skilled in the art as being capable of fulfilling the desired function, for example, plasticizing agents and coalescing agents.

In another embodiment of the invention, the film-forming polymer may be present in the form of particles which are surface-stabilized and dispersed in a liquid fatty phase. For example, the liquid fatty phase comprises a volatile liquid fatty phase, optionally mixed with a non-volatile liquid fatty phase.

The term “volatile fatty phase” is understood to mean any nonaqueous medium capable of evaporating from the skin in less than one hour. This volatile phase comprises, for example, oils having a vapour pressure, at room temperature and atmospheric pressure, ranging from 10⁻³ to 300 mmHg (0.13 Pa to 40,000 Pa).

The liquid fatty phase in which the polymer is dispersed may comprise any physiologically and cosmetically acceptable oils, chosen, for example, from oils of mineral, animal, plant and synthetic origin, carbonaceous, hydrocarbon-based, fluorinated and silicone oils, wherein such oils can form a homogeneous and stable mixture and are compatible with the use envisaged.

The total liquid fatty phase of the composition may represent from 5% to 98% by weight relative to the total weight of the composition, for example, from 20% to 85% by weight relative to the total weight of the composition. The nonvolatile part may represent from 0 to 80%, for example, from 0.1% to 80% by weight of the total weight of the composition, and still further for example from 1% to 50% by weight of the total weight of the composition.

Among the liquid fatty phase that may be used in the invention, mention may be made of fatty acid esters, higher fatty acids, higher fatty alcohols, polydimethylsiloxanes (PDMS), which are optionally phenylated, for example phenyltrimethicones, PDMS that are optionally substituted with aliphatic and/or aromatic groups, which are optionally fluorinated, or with functional groups such as hydroxyl, thiol and amine groups; and polysiloxanes modified with fatty acids, fatty alcoholsor polyoxyalkylenes, fluorinated silicones, and perfluorinated oils.

In one embodiment of the present invention, at least one oil which is volatile at room temperature can be used. After evaporation of the oil, a nonsticky, soft film-forming deposit can be obtained. Furthermore, these volatile oils facilitate the application of the composition to a keratin fibre, such as the eyelashes. These volatile oils may be chosen from, for example, from hydrocarbon oils and silicone oils optionally containing alkyl or alkoxy groups at the end of a silicone-containing or pendant chain.

Mention may be made of such volatile silicone oils that may be chosen from linear and cyclic silicones haivng from 2 to 7 silicon atoms, these silicones optionally comprising at least one alkyl or alkoxy group having from 1 to 10 carbon atoms, for example, octamethylcyclotetrasiloxane, decamethylcyclopentasiloxane, hexadecamethylcyclohexasiloxane, heptamethylhexyltrisiloxane, and heptamethyloctyltrisiloxane.

As volatile hydrocarbon oil, mention may be made of C₈-C₁₆ isoparaffins such as “ISOPARs”, PERMETYLs and for example isododecane.

Such volatile oils may be present in the composition in an amount ranging from 5% to 94.9% by weight of the total weight of the composition, for example, from 20% to 85% by weight of the total weight of the composition.

In one embodiment of the invention, the liquid fatty phase is chosen from:

the nonaqueous liquid compounds having a global solubility parameter according to the HANSEN solubility space of less than 17 (MPa)^{1/2};

the monoalcohols having a global solubility parameter according to the HANSEN solubility space of less than or equal to 20 (MPa)^{1/2}; and

mixtures thereof.

The global solubility parameter global δ according to the HANSEN solubility space is defined in the article “Solubility parameter values” by Eric A. Grulke from the book “Polymer Handbook” 3rd edition, Chapter VII, pages 519-559 by the equation:

δ=(d_{D}²+d_{P}²+d_{H}²)^{1/2}

in which:

d_{D} characterizes the LONDON dispersion forces obtained from the formation of dipoles induced during molecular shocks,

d_{P} characterizes the DEBYE forces of interactions between permanent dipoles,

d_{H} characterizes the specific forces of interactions (hydrogen, acid/base, donor/acceptor type bonds, and the like).

The definition of the solvents in the three-dimensional solubility space according to HANSEN is described in the article by C. M. HANSEN: “The three dimensional solubility parameters” 39 J. Paint Technol. 105 (1967).

Oils which may be used in the liquid fatty phase are, for example, cited in the document EP-A-749747. As a nonaqueous medium, it is also possible to use those described in the document FR-A-2 710 646.

The choice of the nonaqueous medium may be made by persons skilled in the art according to the nature of the monomers constituting the polymer and/or the nature of the stabilizer, as indicated below.

The polymer dispersion may be prepared as described in the document EP-A-749747. The polymerization may be carried out in a dispersion, i.e., by precipitation of the polymer during formation, with protection of the particles formed with a stabilizer.

The polymer particles in dispersion in the said fatty phase can have a size ranging, for example from 5 nm to 600 nm, and further for example from 50 nm to 250 nm, wherein the sizes can be determined by one skilled in the art.

The polymer particles are surface-stabilized using a stabilizer chosen from block polymers, graft polymers, random polymers, and a mixture thereof.

Among the graft polymers, mention may be made of silicone polymers grafted with a hydrocarbon chain and hydrocarbon polymers grafted with a silicone chain.

Also suitable are graft copolymers having, for example, an insoluble backbone of the polyacrylic type with soluble grafts of the poly(12-hydroxystearic) acid type.

It is also possible to use block or graft block copolymers comprising at least one polyorganosiloxane type block and at least one block of a free-radical polymer, for example the graft copolymers of the acrylic/silicone type, which may be used, for example, when the nonaqueous medium contains silicone.

The stabilizer may also be chosen from block and graft block copolymers comprising at least one polyorganosiloxane block and at least one polyether. The polyorganopolysiloxane block may be chosen from, for example, a polydimethylsiloxane and poly(C₂-C₁₈)-alkylmethylsiloxanes; the polyether block may be chosen from, for example, C₂-C₁₈ polyalkylenes, such as polyoxyethylene and polyoxypropylene. Also for example, it is possible to use dimethicone copolyols or (C₂-C₁₈)alkyl methicone copolyols. Further for example, it is possible to use the dimethicone copolyol sold under the name “DOW CORNING 3225C” by the company DOW CORNING, or lauryl methicone copolyol sold under the name “DOW CORNING Q2-5200” by the company DOW CORNING.

Among the block and graft block copolymers, mention may be made of those copolymers comprising at least one block resulting from the polymerization of at least one ethylenic monomer with at least one optionally conjugated ethylenic bond, for example, ethylene, butadiene, and isoprene, and at least one block of a styrene polymer. When the ethylenic monomer comprises several optionally conjugated ethylenic bonds, the residual ethylenic unsaturations after the polymerization are generally hydrogenated. Thus, in a manner known in the art, the polymerization of isoprene leads, after hydrogenation, to the formation of the ethylene-propylene block, and the polymerization of butadiene leads, after hydrogenation, to the formation of the ethylene-butylene block. Among these block copolymers, mention may be made of the “diblock” or “triblock” copolymers, of the polystyrene/polyisoprene or polystyrene/polybutadiene copolymers, for example, those sold under the name “LUVITOL HSB” by BASF; of the polystyrene/copoly(ethylene-propylene) copolymers, for example those sold under the name “KRATON” by Shell Chemical Co; and of the polystyrene/copoly(ethylene-butylene) copolymers.

Mention may also be made, regarding block and graft block copolymers comprising at least one block resulting from the polymerization of at least one ethylenic monomer, for example ethylene, isobutylene, and at least one block of an acrylic polymer, for example methyl methacrylate, of the di- or triblock poly(methyl methacrylate)/polyisobutylene copolymers, and the graft copolymers with a poly(methyl methacrylate) backbone and with polyisobutylene grafts.

As the block and graft block copolymers comprising at least one block resulting from the polymerization of at least one ethylenic monomer and at least one block of a polyether such as a C₂-C₁₈ polyoxyalkylene, for example, polyoxyethylene and polyoxypropylene, di- or triblock polyoxyethylene/polybutadiene or polyoxy-ethylene/polyisobutylene copolymers may be used.

It is also possible to use copolymers of C₁-C₄ alkyl (meth)acrylates and C₈-C₃₀ alkyl (meth)acrylates, for example, the stearyl methacrylate/methyl methacrylate copolymer.

In this case, a graft polymer or a block polymer can be used as a stabilizer, so as to have a better interfacial activity. Indeed, the blocks or grafts insoluble in the synthesis solvent provide a more voluminous covering at the surface of the particles.

When the liquid fatty phase comprises at least one silicone oil, the stabilizing agent may, for example, be chosen from block and graft block copolymers comprising at least one block of the polyorganosiloxane type and at least one block of a free-radical polymer, of a polyether or of a polyester, for example, polyoxy(C₂-C₈)alkylenes, such as polyoxypropylenated and oxyethylenated blocks.

When the liquid fatty phase does not comprise a silicone oil, the stabilizing agent can be, for example, chosen from:

(a) block and graft block copolymers comprising at least one block of the polyorganosiloxane type and at least one block of a free-radial polymer, of a polyether or of a polyester,

(b) copolymers of C₁-C₄ alkyl acrylates or methacrylates, and C₈-C₃₀ alkyl acrylates or methacrylates,

(c) block and graft block copolymers comprising at least one block resulting from the polymerization of at least one ethylenic monomer, with conjugated ethylenic bonds, and at least one block of vinyl and acrylic polymers, of polyethers and of polyesters.

Diblock polymers can be, for example, used as the stabilizing agent.

In yet another embodiment of the invention, the film-forming polymer may be present in a solubilized form in a liquid fatty phase as defined above, also called a fat-soluble polymer.

For example, the fat-soluble polymer includes, but is not limited to those polymers corresponding to the following formula (II):

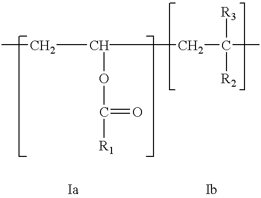

wherein:

R₁ is chosen from saturated, linear and branched, hydrocarbon chains having from 1 to 19 carbon atoms;

R₂ is a radical chosen from radicals of:

a) —O—CO—R₄, wherein R₄ has the same definition as R₁, except that R₁ and R₄ must be different when in the same copolymer;

b) —CH₂—R₅, wherein R₅ is chosen from saturated, linear and branched, hydrocarbon chains having from 5 to 25 carbon atoms;

c) —O—R₆, wherein R₆ is chosen from saturated hydrocarbon chains having from 2 to 18 carbon atoms; and

d) —CH₂—O—CO—R₇, wherein R₇ is chosen from saturated, linear and branched, hydrocarbon chains having from 1 to 19 carbon atoms,

R₃ represents a hydrogen atom when R₂ is chosen from the radicals a), b) and c), or R₃ represents a methyl radical when R₂ is chosen from the radicals d),

it being necessary that the said copolymer comprise at least 15% by weight of at least one monomer derived from unit (Ia) or from unit (Ib) in which the saturated or branched hydrocarbon chains have at least 7 carbon atoms.

The copolymers of formula (II) result from the copolymerization of at least one vinyl ester (corresponding to the unit (Ia)) and of at least one other monomer (corresponding to the unit (Ib)) which may be chosen from α-olefins, alkyl vinyl ethers, and allyl and methallyl esters.

When, in the unit (Ib), R₂ is chosen from the radicals —CH₂—R₅, —O—R₆ and —CH₂—O—CO—R₇ as defined above, the copolymer of formula (II) may comprise 50 to 95 mol % of at least one unit (Ia) and 5 to 50 mol % of at least one unit (Ib).

The copolymers of formula (II) may also result from the copolymerization of at least one first vinyl ester and at least one other vinyl ester different from the first. In this case, these copolymers may comprise 10 to 90 mol % of at least one unit (Ia) and 10 to 90 mol % of at least one unit (Ib) wherein R₂ is chosen from the radicals —O—CO—R₄.

Among the vinyl esters leading to the unit of formula (Ia), or to the unit of formula (Ib) in which R₂=—O—CO—R₄, mention may be made of vinyl acetate, vinyl propionate, vinyl butanoate, vinyl octanoate, vinyl decanoate, vinyl laurate, vinyl stearate, vinyl isostearate, vinyl 2,2-dimethyloctanoate and vinyl dimethylpropionate.

Among the α-olefins leading to the unit of formula (Ib) in which R₂=—CH₂—R₅, mention may be made of 1-octene, 1-dodecene, 1-octadecene, 1-eicosene and mixtures of α-olefins having from 22 to 28 carbon atoms.

Among the alkyl vinyl ethers leading to the unit of formula (Ib) in which R₂=—O—R₆, mention may be made of ethyl vinyl ether, n-butyl vinyl ether, isobutyl vinyl ether, decyl vinyl ether, dodecyl vinyl ether, cetyl vinyl ether and octadecyl vinyl ether.

Among the allyl and methallyl esters leading to the unit of formula (Ib) in which R₂=—CH₂—O—CO—R₇, mention may be made of allyl and methallyl acetates, propionates, dimethylpropionates, butyrates, hexanoates, octanoates, decanoates, laurates, 2,2-dimethylpentanoates, stearates and eicosanoates.

The copolymers of formula (II) may also be crosslinked using certain types of crosslinking agents which are intended to substantially increase their molecular weight.

Such crosslinking can be carried out during the copolymerization and the crosslinking agents may be chosen from vinyls, allyls and methallyls. Among these crosslinking agents, mention may be made, for example, of tetraallyloxyethane, divinylbenzene, divinyl octanedioate, divinyl dodecanedioate and divinyl octadecanedioate.

Among the various copolymers of formula (II) which can be used in the composition according to the invention, mention may be made, for example, of the copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl propionate/vinyl stearate, allyl dimethyl propionate/vinyl stearate, vinyl propionate/vinyl stearate, crosslinked with 0.2% of divinylbenzene, vinyl dimethyl propionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene crosslinked with 0.2% of divinylbenzene and allyl propionate/allyl stearate crosslinked with 0.2% of divinylbenzene.

Among the fat-soluble film-forming polymers, mention may be made of fat-soluble homopolymers, for example, those resulting from the homopolymerization of vinyl esters having from 9 to 22 carbon atoms, or of alkyl acrylates or methacrylates, the alkyl radicals having from 10 to 20 carbon atoms.

Such fat-soluble homopolymers may be chosen from polyvinyl stearate, polyvinyl stearate crosslinked using divinylbenzene, diallyl ether, and diallyl phthalate, polystearyl (meth)acrylate, polyvinyl laurate, and polylauryl (meth)acrylate, it being possible for these poly(meth)acrylates to be crosslinked using ethylene glycol or tetraethylene glycol dimethacrylate.

The fat-soluble copolymers and homopolymers defined above are known in the art and, for example, are described in document FR-A-2232303. Such fat-soluble copolymers and homopolymers may have a weight average molecular weight ranging, for example, from 2000 to 500,000, and further, for example, from 4000 to 200,000.

Among the fat-soluble film-forming polymers which can be used in accordance with the invention, mention may be made of polyalkylenes, for example, copolymers of C₂-C₂₀ alkenes, different from the polyolefin wax defined in unit (Ia), for example, polybutene; alkyl celluloses with a saturated or unsaturated, linear or branched, C₁ to C₈ alkyl radical such as ethyl cellulose or propyl cellulose; copolymers of vinylpyrrolidone (VP) for example, copolymers of vinylpyrrolidone and of a C₂ to C₄₀, and such as C₃ to C₂₀, alkenes. By way of example of the VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene and VP/acrylic acid/lauryl methacrylate copolymers.

The film-forming polymer may be present in the form of a dry matter content ranging, for example, from 5% to 60% by weight relative to the total weight of the composition, and also, for example, from 10% to 45% by weight, and further for example—from 15% to 35% by weight relative to the total weight of the composition.

The said ionic surfactant and the hydrophobic film-forming polymer may be present in the composition in a hydrophobic film-forming polymer/ionic surfactant weight ratio ranging, for example, from 30:1 to 0.1:1, and also for example from 15:1 to 0.5:1, and further for example from 8:1 to 1:1.

The composition may further comprise at least one colouring material chosen from pulverulent compounds, and fat-soluble and water-soluble colorants, for example, in an amount ranging from 0.01% to 50% by weight of the total weight of the composition. The pulverulent compounds may be chosen from the pigments and the pearlescent agents that are normally used in cosmetic compositions. The pulverulent compounds may represent, for example from 0.1 to 25% by weight of the total weight of the composition and further for example from 1 to 20% by weight of the total weight of the composition.

The pigments may be white or coloured, inorganic and/or organic. Among the inorganic pigments, mention may be made of titanium dioxide, optionally surface-treated, zirconium or cerium oxides, as well as iron or chromium oxides, manganese violet, ultramarine blue, chromium hydrate and ferric blue. Among the organic pigments, mention may be made of carbon black, pigments of the D & C type, and lacquers based on carmine, barium, strontium, calcium and aluminium.

The pearlescent pigments may be chosen from white pearlescent pigments, for example mica coated with titanium or with bismuth oxychloride; coloured pearlescent pigments, for example, mica-titanium with iron oxides, mica-titanium with, for example, ferric blue or chromium oxide, mica-titanium with an organic pigment of the abovementioned type as well as pearlescent pigments based on bismuth oxychloride.

The composition may also comprise fillers which may be chosen from those well known to persons skilled in the art and which are commonly used in cosmetic compositions. The fillers may be inorganic or organic, lamellar or spherical. Among the fillers, mention may be made of talc, mica, silica, kaolin, nylon (Orgasol from Atochem), poly-β-alanine and polyethylene powders, Teflon, lauroyl-lysine, starch, boron nitride, powders of tetrafluoroethylene polymers, hollow microspheres such as Expancel (Nobel Industrie), polytrap (Dow Corning) and microbeads of silicone resin (Tospearls from Toshiba, for example), precipitated calcium carbonate, magnesium carbonate and hydrocarbonate, hydroxyapatite, hollow microspheres of silica (SILICA BEADS from MAPRECOS), glass or ceramic microcapsules, metallic soaps derived from carboxylic organic acids having, for example, from 8 to 22 carbon atoms, and further for example from 12 to 18 carbon atoms, for example zinc, magnesium or lithium stearate, zinc laurate or magnesium myristate.

The composition may further comprise any additive customarily used in such compositions, for example, thickeners, preservatives, perfumes, sunscreens, anti-free radical agents, waxes, oils, moisturizing agents, vitamins, proteins, plasticizers, sequestrants, ceramides, alkalinizing or acidifying agents, and emollients.

Of course, persons skilled in the art would be careful to choose such optional additional compounds, and their quantity, so that the advantageous properties of the composition according to the invention are not, or not substantially, impaired by the addition envisaged.

Another aspect of the invention is the use of a composition as defined above for obtaining a film deposited on a keratin material, which may be resistant to cold water and/or which can be removed as make-up with hot water.

Yet another aspect of the invention is a cosmetic method for the application of make-up to, or the non-therapeutic care of, a keratin material comprising applying to the keratin material a composition as defined above.

Still another aspect of the invention is a cosmetic method for removing make-up from a keratin material to which a composition as defined above has been applied as make-up, comprising at least one rinsing once said keratin material to which make-up has been applied with hot water heated to a temperature greater than or equal to 35° C.

The invention is illustrated in greater detail in the following non-limiting examples.

### EXAMPLES 1 and 2

Mascaras having the following composition were prepared:

| | |
|---|---|
| polyurethane as an aqueous dispersion sold under | 21 g AS |
| the name AVALURE UR 425 by the company | |
| GOODRICH containing 49% by weight of active | |
| substances (AS) | |
| hydroxyethyl cellulose | 1.9 g |
| anionic surfactant | 3 g |
| black iron oxide | 5 g |
| propylene glycol | 5 g |
| preservatives | qs |
| water | qs 100 g |

For each composition, a layer 300 μm thick (before drying) was spread over a glass slab and allowed to dry for 24 hours at 30° C. and 50% relative humidity. After drying, the slab was placed in a crystallizing vessel containing water at room temperature (RT) (20° C.) or at 40° C. and then a magnetic bar was placed on the film. The bar was stirred using a magnetic stirrer and the time (expressed in minutes) after which the film starts to disintegrate is measured.

The following results were obtained:

| Example | 1 | 2 |
|---|---|---|
| Ionic surfactant | sodium stearate | sodium |
| | | N-octadecylphthalamate |
| 20° C. | >2 hours | >2 hours |
| 40° C. | 10 minutes | 16 minutes |

It was observed that, for each composition, the film was less resistant in the presence of water at 40° C. (hot water) than in the presence of water at room temperature (cold water). The film can therefore be more easily removed as make-up with hot water and is more resistant to cold water.

### EXAMPLE 3:

A mascara having the following composition was prepared:

| | |
|---|---|
| polyurethane as an aqueous dispersion sold under | 16 g AS |
| the name AVALURE UR 425 by the company | |
| GOODRICH containing 49% by weight of active | |
| substances (AS) | |
| hydroxyethyl cellulose | 1.9 g |
| sodium N-octadecyl phthalamate | 0.6 g |
| beeswax | 6 g |
| black iron oxide | 5 g |
| propylene glycol | 5 g |
| preservatives | qs |
| water | qs 100 g |

In this composition, the sodium N-octadecylpthalamate may be replaced with sodium stearate.

This mascara was easy to apply to the eyelashes and the make-up obtained exhibited good resistance to cold water and was easy to remove as make-up with hot water.

What is claimed is:

1. A make-up composition for deposit on a keratin material, comprising, in said composition, a physiologically acceptable medium, at least one hydrophobic film-forming polymer, and at least one ionic surfactant exhibiting a variation in conductivity in water, at a concentration of 2% by weight in water, of at least 100 μS/cm in a temperature range of from 20° C. to 45° C., wherein said deposited composition is removable from the keratin material with hot water.

2. The composition according to claim 1, said at least one ionic surfactant exhibiting a variation in conductivity in water, at a concentration of 2% by weight in water, of at least 100 μS/cm in a temperature range of from 30° C. to 45° C.

3. The composition according to claim 2, said at least one ionic surfactant exhibiting a variation in conductivity in water, at a concentration of 2% by weight in water, of at least 100 μS/cm in a temperature range of from 40° C. to 45° C.

4. The composition according to claim 1, said at least one ionic surfactant exhibiting a variation in conductivity ranging from 100 to 2000 μS/cm.

5. The composition according to claim 4, said at least one ionic surfactant exhibiting a variation in conductivity ranging from 200 to 1500 μS/cm.

6. The composition according to claim 5, said at least one ionic surfactant exhibiting a variation in conductivity ranging from 200 to 1000 μS/cm.

7. The composition according to claim 1, wherein the at least one ionic surfactant is chosen from anionic surfactants.

8. The composition according to claim 1, wherein the at least one ionic surfactant is chosen from sodium stearate, and phthalamates of formula (I): 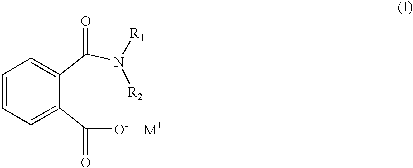
wherein,
R₁ is chosen from alkyl radicals having from 16 to 20 carbon atoms,
R₂ is chosen from H and —CH₃,
M is chosen from H, Na, K, NH₄, (HOCH₃CH₂)₃NH, and (HOCH₃CH₂)₂NH₂.

9. The composition according to claim 8, wherein R₁ is chosen from alkyl radicals having 18 carbon atoms.

10. The composition according to claim 8, wherein R₂ is a hydrogen atom.

11. The composition according to claim 8, wherein the at least one ionic surfactant is chosen from sodium stearate and sodium N-octadecylphthalamate.

12. The composition according to claim 1, said at least one ionic surfactant exhibiting a variation in conductivity in water, at a concentration of 2% by weight in water, of at least 400 μS/cm in a temperature range of from 20° C. to 45° C.

13. The composition according to claim 12, said at least one ionic surfactant exhibiting a variation in conductivity in water, at a concentration of 2% by weight in water, of at least 400 μS/cm in a temperature range of from 30° C. to 45° C.

14. The composition according to claim 13, said at least one ionic surfactant exhibiting a variation in conductivity in water, at a concentration of 2% by weight in water, of at least 400 μS/cm in a temperature range of from 40° C. to 45° C.

15. The composition according to claim 11, wherein the at least one ionic surfactant is sodium N-octadecylphthalamate.

16. The composition according to claim 1, wherein the at least one ionic surfactant has a molecular weight ranging from 200 to 1000.

17. The composition according to claim 16, wherein the at least one ionic surfactant has a molecular weight ranging from 250 to 800.

18. The composition according to claim 17, wherein the at least one ionic surfactant has a molecular weight ranging from 250 to 600.

19. The composition according to claim 1, wherein said at least one ionic surfactant is present in an amount ranging from 0.1% to 20% by weight relative to the total weight of the composition.

20. The composition according to claim 19, wherein said at least one ionic surfactant is present in an amount ranging from 0.5% to 15% by weight relative to the total weight of the composition.

21. The composition according to claim 20, wherein said at least one ionic surfactant is present in an amount ranging from 1% to 10% by weight relative to the total weight of the composition.

22. The composition according to claim 1, further comprising glyceryl monostearate.

23. The composition according to claim 22, wherein the glyceryl monostearate is present in an amount ranging from 0.1% to 5% by weight relative to the total weight of the composition.

24. The composition according to claim 23, wherein the glyceryl monostearate is present in an amount ranging from 0.5% to 2% by weight relative to the total weight of the composition.

25. The composition according to claim 22, wherein the at least one ionic surfactant and the glyceryl monostearate are present in the composition in an ionic surfactant/glyceryl monostearate weight ratio ranging from 0.01:1 to 2:1.

26. The composition according to claim 25, wherein the at least one ionic surfactant and the glyceryl monostearate are present in the composition in an ionic surfactant/glyceryl monostearate weight ratio ranging from 0.5:1 to 2:1.

27. The composition according to claim 26, wherein the at least one ionic surfactant and the glyceryl monostearate are present in the composition in an ionic surfactant/glyceryl monostearate weight ratio ranging from 1:1 to 2:1.

28. The composition according to claim 1, wherein the at least one hydrophobic film-forming polymer is chosen from free-radical polymers, polycondensates, and polymers of natural origin.

29. The composition according to claim 28, wherein the at least one hydrophobic film-forming polymer is chosen from vinyl polymers, polyurethanes, polyesters, and cellulosic polymers.

30. The composition according to claim 1, wherein the at least one hydrophobic film-forming polymer is present in the form of particles dispersed in an aqueous medium.

31. The composition according to claim 30, wherein the at least one hydrophobic film-forming polymer is a polyurethane in the form of particles in aqueous dispersion.

32. The composition according to claim 1, wherein the at least one hydrophobic film-forming polymer is present in the form of particles dispersed in a liquid fatty phase and surface-stabilized.

33. The composition according to claim 32, wherein the particles of the at least one hydrophobic film-forming polymer are stabilized by at least one stabilizer chosen from block polymers, graft polymers, and random polymers.

34. The composition according to claim 33, wherein the at least one stabilizer is chosen from block and graft block polymers comprising at least one block resulting from the polymerization of at least one ethylene monomer comprising at least one optionally conjugated ethylene bond and at least one block of a styrene polymer.

35. The composition according to claim 1, wherein the at least one hydrophobic film-forming polymer is present in the form of a dry matter content ranging from 5% to 60% by weight relative to the total weight of the composition.

36. The composition according to claim 35, wherein the at least one hydrophobic film-forming polymer is present in the form of a dry matter content ranging from 10% to 45% by weight relative to the total weight of the composition.

37. The composition according to claim 1, wherein the at least one hydrophobic film-forming polymer and the at least one ionic surfactant are present in the composition in a hydrophobic film-forming polymer/ionic surfactant weight ratio ranging from 0.1:1 to 30:1.

38. The composition according to claim 37, wherein the at least one hydrophobic film-forming polymer and the at least one ionic surfactant are present in the composition in a hydrophobic film-forming polymer/ionic surfactant weight ratio ranging from 0.5:1 to 15:1.

39. The composition according to claim 38, wherein the at least one hydrophobic film-forming polymer and the at least one ionic surfactant are present in the composition in a hydrophobic film-forming polymer/ionic surfactant weight ratio ranging from 1:1 to 8:1.

40. The composition according to claim 1, further comprising at least one cosmetic additive chosen from colouring matter, fillers, thickeners, preservatives, perfumes, sunscreens, anti-free radical agents, waxes, oils, moisturizing agents, vitamins, proteins, plasticizers, sequestrants, ceramides, alkalinizing and acidifying agents, and emollients.

41. The composition according to claim 1, wherein the composition is provided in the form of a mascara, an eyeliner, a product for lip, a blusher, an eyeshadow, a foundation, a make-up product for a body, a concealer, a product for nail, a composition for protecting against sunlight or for colouring skin, or a care product for skin.

42. A mascara for deposit on a keratin material, comprising, in said mascara, a physiologically acceptable medium, at least one hydrophobic film-forming polymer, and at least one ionic surfactant exhibiting a variation in conductivity in water, at a concentration of 2% by weight in water, of at least 100 μS/cm in a temperature range of from 20° C. to 45° C., wherein said deposited mascara is removable from the keratin material with hot water.

43. A cosmetic composition comprising, in a cosmetically acceptable medium, at least one hydrophobic film-forming polymer and at least one ionic surfactant of formula (I): 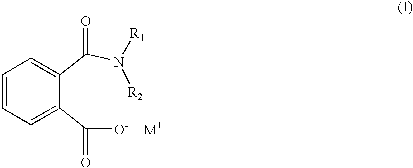
wherein,
R₁ is chosen from alkyl radicals having from 16 to 20 carbon atoms,
R₂ is chosen from H and —CH₃,
M is chosen from H, Na, K, NH₄, (HOCH₂CH₂)₃NH, and (HOCH₂CH₂)₂NH₂.

44. The cosmetic composition according to claim 43, wherein R₁ is chosen from alkyl radicals having 18 carbon atoms.

45. The cosmetic composition according to claim 43, wherein R₂ is a hydrogen atom.

46. The cosmetic composition according to claim 43, wherein the at least one ionic surfactant is sodium N-octadecylphthalamate.

47. The cosmetic composition according to claim 43, wherein the at least one ionic surfactant is present in an amount ranging from 0.1% to 20% by weight relative to the total weight of the composition.

48. The cosmetic composition according to claim 47, wherein the at least one ionic surfactant is present in an amount ranging from 0.5% to 15% by weight relative to the total weight of the composition.

49. The cosmetic composition according to claim 48, wherein the at least one ionic surfactant is present in an amount ranging from 1% to 10% by weight relative to the total weight of the composition.

50. The cosmetic composition according to claim 43, further comprising glyceryl monostearate.

51. The cosmetic composition according to claim 50, wherein the glyceryl monostearate is present in an amount ranging from 0.1% to 5% by weight relative to the total weight of the composition.

52. The cosmetic composition according to claim 51, wherein the glyceryl monostearate is present in an amount ranging from 0.2% to 1% by weight relative to the total weight of the composition.

53. The cosmetic composition according to claim 50, wherein the at least one ionic surfactant and the glyceryl monostearate are present in the composition in an ionic surfactant/glyceryl monostearate weight ratio ranging from 0.01:1 to 2:1.

54. The cosmetic composition according to claim 53, wherein the at least one ionic surfactant and the glyceryl monostearate are present in the composition in an ionic surfactant/glyceryl monostearate weight ratio ranging from 0.5:1 to 2:1.

55. The cosmetic composition according to claim 54, wherein the at least one ionic surfactant and the glyceryl monostearate are present in the composition in an ionic surfactant/glyceryl monostearate weight ratio ranging from 1:1 to 2:1.

56. The cosmetic composition according to claim 43, wherein the at least one hydrophobic film-forming polymer is chosen from free-radical polymers, polycondensates, and polymers of natural origin.

57. The cosmetic composition according to claim 56, wherein the at least one hydrophobic film-forming polymer is chosen from vinyl polymers, polyurethanes, polyesters, and cellulosic polymers.

58. The cosmetic composition according to claim 43, wherein the at least one hydrophobic film-forming polymer is present in the form of particles dispersed in an aqueous medium.

59. The cosmetic composition according to claim 58, wherein the at least one hydrophobic film-forming polymer is a polyurethane in the form of particles in aqueous dispersion.

60. The cosmetic composition according to claim 43, wherein the at least one hydrophobic film-forming polymer is present in the form of particles dispersed in a liquid fatty phase and surface-stabilized.

61. The cosmetic composition according to claim 60, wherein the particles of the at least one hydrophobic film-forming polymer are stabilized by at least one stabilizer chosen from block polymers, graft polymers, and random polymers.

62. The cosmetic composition according to claim 61, wherein the at least one stabilizer is chosen from block and graft block polymers comprising at least one block resulting from the polymerization of at least one ethylene monomer comprising at least one optionally conjugated ethylene bond and at least one block of a styrene polymer.

63. The cosmetic composition according to claim 43, wherein the at least one hydrophobic film-forming polymer is present in the form of dry matter content ranging from 5% to 60% by weight relative to the total weight of the composition.

64. The cosmetic composition according to claim 63, wherein the at least one hydrophobic film-forming polymer is present in the form of dry matter content ranging from 10% to 45% by weight relative to the total weight of the composition.

65. The cosmetic composition according to claim 43, wherein the at least one hydrophobic film-forming polymer and the at least one ionic surfactant are present in the composition in a hydrophobic film-forming polymer/ionic surfactant weight ratio ranging from 0.1:1 to 30:1.

66. The cosmetic composition according to claim 65, wherein the at least one hydrophobic film-forming polymer and the at least one ionic surfactant are present in the composition in a hydrophobic film-forming polymer/ionic surfactant weight ratio ranging from 0.5:1 to 20:1.

67. The cosmetic composition according to claim 66, wherein the at least one hydrophobic film-forming polymer and the at least one ionic surfactant are present in the composition in a hydrophobic film-forming polymer/ionic surfactant weight ratio ranging from 1:1 to 8:1.

68. The cosmetic composition according to claim 43, further comprising at least one additive chosen from thickeners, fat-soluble and water-soluble colorants, preservatives, perfumes, sunscreens, anti-free radical agents, waxes, oils, moisturizing agents, vitamins, proteins, plasticizers, sequestrants, ceramides, alkalinizing and acidifying agents, and emollients.

69. The cosmetic composition according to claim 43, wherein the composition is provided in the form of a mascara, an eyeliner, a product for lips, a blusher, an eyeshadow, a foundation, a make-up product for a body, a concealer, a product for nails, a composition for protecting against sunlight or for colouring skin, or a care product for skin.

70. A mascara comprising, in said mascara, a cosmetically acceptable medium, at least one hydrophobic film-forming polymer, and at least one ionic surfactant of formula (I): 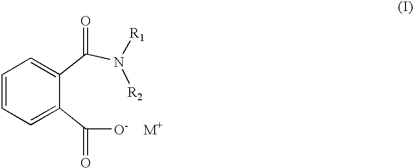
wherein,
R₁ is chosen from alkyl radicals having from 16 to 20 carbon atoms,
R₂ is chosen from H and —CH₃,
M is chosen from H, Na, K, NH₄, (HOCH₂CH₂)₃NH, and (HOCH₂CH₂)₂NH₂.

71. A method for application of a make-up to, or non-therapeutic care of, a keratin material comprising depositing on the keratin material a composition comprising, in a physiologically acceptable medium, at least one hydrophobic film-forming polymer and at least one ionic surfactant exhibiting a variation in conductivity in water, at a concentration of 2% by weight in water, of at least 100 μS/cm in a temperature range of from 20° C. to 45° C., wherein the deposited composition is removable from the keratin material with hot water.

72. A method for application of a make-up to, or non-therapeutic care of, a keratin material comprising depositing on the keratin material a composition comprising, in a cosmetically acceptable medium, at least one hydrophobic film-forming polymer and at least one ionic surfactant of formula (I): 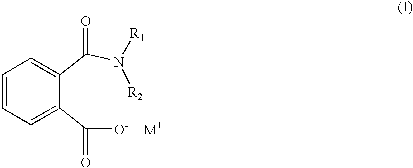
wherein,
R₁ is chosen from alkyl radicals having from 16 to 20 carbon atoms,
R₂ is chosen from H and —CH₃,
M is chosen from H, Na, K, NH₄, (HOCH₂CH₂)₃NH, and (HOCH₂CH₂)₂NH₂.

73. A method for making up a keratin material comprising depositing on the keratin material a make up composition comprising, in a physiologically acceptable medium, at least one hydrophobic film-forming polymer and at least one ionic surfactant exhibiting a variation in conductivity in water, at a concentration of 2% by weight in water, of at least 100 μS/cm in a temperature range of from 20° C. to 45° C., wherein the deposited make up composition is resistant to cold water and/or is removable from the keratin material with hot water.

74. A method for making up a keratin material comprising depositing on the keratin material a make up composition comprising, in a cosmetically acceptable medium, at least one hydrophobic film-forming polymer and at least one ionic surfactant of formula (I): 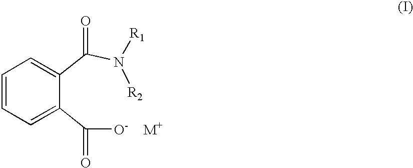
wherein,
R₁ is chosen from alkyl radicals having from 16 to 20 carbon atoms,
R₂ is chosen from H and —CH₃,
M is chosen from H, Na, K, NH₄, (HOCH₂CH₂)₃NH, and (HOCH₂CH₂)₂NH₂,
wherein the deposited make up composition is resistant to cold water and/or is removable from the keratin material with hot water.

75. A method for removing make-up from a keratin material,
comprising rinsing at least once, with hot water at a temperature of from 35° C. to 50° C., a keratin material bearing a deposit of a make-up comprising a physiologically acceptable medium, at least one hydrophobic film-forming polymer, and at least one ionic surfactant exhibiting a variation in conductivity in water, at a concentration of 2% by weight in water, of at least 100 μS/cm in a temperature range of from 20° C. to 45° C.

76. A method for removing make-up from a keratin material,
comprising rinsing at least once, with hot water at a temperature of from 35° C. to 50° C., a keratin material bearing a deposit of a make-up comprising a physiologically acceptable medium, at least one hydrophobic film-forming polymer, and at least one ionic surfactant of formula (I): 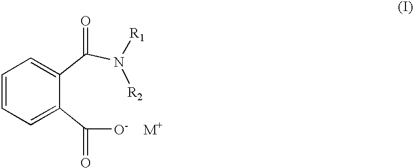
wherein,
R₁ is chosen from alkyl radicals having from 16 to 20 carbon atoms,
R₂ is chosen from H and —CH₃,
M is chosen from H, Na, K, NH₄, (HOCH₂CH₂)₃NH, and (HOCH₂CH₂)₂NH₂.

77. The method according to claim 75, wherein the hot water contains no detergent agent.

78. The method according to claim 76, wherein the hot water contains no detergent agent.

79. A process for manufacturing a cosmetic composition for deposit on a keratin material comprising including in the composition at least one hydrophobic film-forming polymer, and at least one ionic surfactant exhibiting a variation in conductivity in water, at a concentration of 2% by weight in water, of at least 100 μS/cm in a temperature range of from 20° C. to 45° C., wherein said deposit is removable from the keratin material with hot water.

80. A process for manufacturing a cosmetic composition comprising including in the composition at least one hydrophobic film-forming polymer, and at least one ionic surfactant of formula (I): 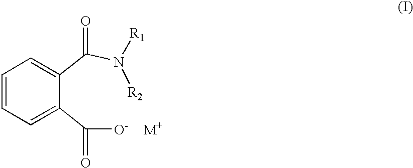
wherein,
R₁ is chosen from alkyl radicals having from 16 to 20 carbon atoms,
R₂ is chosen from H and —CH₃,
M is chosen from H, Na, K, NH₄, (HOCH₂CH₂)₃NH, and (HOCH₂CH₂)₂NH₂.
